(19) United States
(12) Patent Application Publication
(10) Pub. No.: US 2023/0102303 A1
(43) Pub. Date: Mar. 30, 2023

(54) MEDICAL IMAGE PROCESSING APPARATUS AND MEDICAL IMAGE PROCESSING METHOD

(71) Applicants: Royal Melbourne Hospital Neuroscience Foundation, Parkville (AU); CANON MEDICAL SYSTEMS CORPORATION, Tochigi (JP)

(72) Inventors: Mark PARSONS, East Hills (AU); Andrew BIVARD, Parkville (AU); Ryota MATSUBARA, Nasushiobara (JP); Takashi SHIGETA, Nasushiobara (JP); Takuya SAKAGUCHI, Utsunomiya (JP)

(73) Assignees: Royal Melbourne Hospital Neuroscience Foundation, Parkville (AU); CANON MEDICAL SYSTEMS CORPORATION, Tochigi (JP)

(21) Appl. No.: 17/935,362

(22) Filed: Sep. 26, 2022

(30) Foreign Application Priority Data
Sep. 30, 2021 (JP) 2021-162181

(51) Int. Cl. A61B 6/00; A61B 6/03

(52) U.S. Cl. CPC A61B 6/507; A61B 6/03; A61B 6/5217

(57) ABSTRACT

A medical image processing apparatus according to an embodiment includes processing circuitry. The processing circuitry acquires medical images of multiple time phases. The processing circuitry generates a vascular territory image showing plural vascular territories included in the subject tissue. The processing circuitry sets a region of interest in the subject tissue. The processing circuitry sets at least two regions out of the vascular territories and an ischemia area in the region of interest based on the vascular territory image. The processing circuitry calculates a ratio of each of the at least two regions to the region of interest. The processing circuitry outputs information about the ratio.

### CROSS-REFERENCE TO RELATED APPLICATIONS

This application is based upon and claims the benefit of priority from Japanese Patent Application No. 2021-162181, filed on Sep. 30, 2021; the entire contents of which are incorporated herein by reference.

### FIELD

Embodiments disclosed in the present specification and the drawings relate to a medical image processing apparatus and a medical image processing method.

### BACKGROUND

Conventionally, diagnosis of infarction of a subject tissue of a brain and the like has been performed by using a medical image processing apparatus. For patients with an infarction, because presence of a collateral circulation is an indicator for a possibility of recovery of a tissue, its evaluation is important. The collateral circulation is a circulation vessel that is newly developed, when a stenosis or an occlusion occurs, to compensate a blood flow deficit caused thereby. Therefore, it is desired to quantitatively evaluate collateral circulation that is directly connected to a treatment decision and a prognosis at the tissue level.

### BRIEF DESCRIPTION OF THE DRAWINGS

FIG. 1 is a diagram illustrating an example of a configuration of a medical image processing apparatus according to a first embodiment;

FIGS. 2A, 2B, 2C, and 2D are diagrams for explaining an example of processing performed by respective processing functions included in processing circuitry according to the first embodiment;

FIG. 3 is a flowchart illustrating a procedure of processing performed by the processing circuitry according to the first embodiment;

FIGS. 4A, 4B, 4C, and 4D are diagrams for explaining an example of processing performed by respective processing functions included in processing circuitry according to a second embodiment;

FIGS. 5A, 5B, 5C, 5D, and 5E are diagrams for explaining an example of processing performed by respective processing functions included in processing circuitry according to a third embodiment;

FIGS. 6A, 6B, 6C, 6D, and 6E are diagrams for explaining an example of processing performed by respective processing functions included in processing circuitry according to a fourth embodiment.

FIGS. 7A, 7B, 7C, and 7D are diagrams for explaining an example of processing performed by respective processing functions included in processing circuitry according to a fifth embodiment;

FIGS. 8A, 8B, 8C, and 8D are diagrams for explaining an example of processing performed by respective processing functions included in processing circuitry according to a sixth embodiment;

FIG. 9 is a diagram illustrating an example of a configuration of a medical image processing apparatus according to a seventh embodiment;

FIG. 10 is a diagram for explaining an example of processing performed by a determining function included in processing circuitry according to the seventh embodiment; and

FIG. 11 is a flowchart illustrating a procedure of processing performed by the processing circuitry according to the seventh embodiment.

### DETAILED DESCRIPTION

A medical image processing apparatus according to an embodiment includes an acquiring unit, a generating unit, a first setting unit, a second setting unit, a calculating unit, and an output unit. The acquiring unit acquires medical images of multiple time phases of a subject tissue of a subject. The generating unit generates a vascular territory image showing plural vascular territories included in the subject tissue based on the medical images of multiple time phases. The first setting unit sets a region of interest in the subject tissue. The second setting unit sets at least two regions out of the vascular territories and an ischemia area in the region of interest based on the vascular territory image. The calculating unit calculates a ratio of each of the at least two regions and the region of interest. The output unit outputs information relating to the ratio.

Hereinafter, embodiments of a medical image processing apparatus and a medical image processing method will be explained in detail with reference to the drawings.

### First Embodiment

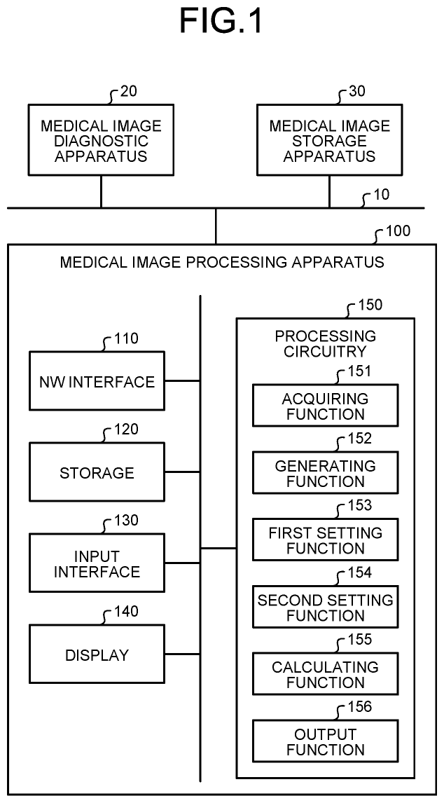

FIG. 1 is a diagram illustrating an example of a configuration of a medical image processing apparatus according to a first embodiment.

For example, as illustrated in FIG. 1, a medical image processing apparatus 100 according to the present embodiment is connected to a medical image diagnostic apparatus 20 and a medical image storage apparatus 30 through a network 10 such that mutual communication is enabled.

The medical image diagnostic apparatus 20 acquires a medical image of a subject (patient or the like) to be used for image-based diagnosis and the like. Specifically, the medical image diagnostic apparatus 20 generates a two-dimensional image or a three-dimensional image (also called volume data) of a subject as the medical image. For example, the medical image diagnostic apparatus 20 is an X-ray computed tomography (CT) apparatus, a magnetic resonance imaging (MRI) apparatus, or the like.

The medical image storage apparatus 30 acquires the medical image from the medical image diagnostic apparatus 20 through the network 10, and stores the acquired medical image in a storage in the medical image storage apparatus 30. For example, the medical image storage apparatus 30 is implemented by a computer device, such as a server and a workstation.

The medical image processing apparatus 100 acquires a medical image from the medical image diagnostic apparatus 20 or the medical image storage apparatus 30 through the network 10, and performs various kinds of processing by using the acquired medical image. For example, the medical image processing apparatus 100 is implemented by a computer device, such as a server, a workstation, a personal computer, and a tablet terminal.

Specifically, the medical image processing apparatus 100 includes a network (NW) interface 110, a storage 120, an input interface 130, a display 140, and processing circuitry 150.

The NW interface 110 controls transmission and communication of various kinds of data transmitted and received between other devices connected through the network 10 and the medical image processing apparatus 100. Specifically, the NW interface 110 is connected to the processing circuitry 150, and outputs a medical image received from the medical image diagnostic apparatus 20 or the medical image storage apparatus 30 to the processing circuitry 150. For example, the NW interface 110 is implemented by a network card, a network adopter, a network interface controller (NIC), and the like.

The storage 120 stores various kinds of data, various kinds of programs, and the like. Specifically, the storage 120 is connected to the processing circuitry 150, and stores an input medical image or outputs a stored medical image to the processing circuitry 150 in response to a command sent from the processing circuitry 150. For example, the storage 120 is implemented by a semiconductor memory device, such as a random access memory (RAM) and a flash memory, a hard disk, an optical disk, or the like.

The input interface 130 accepts an input operation of various kinds of instructions and various kinds of information from an operator. Specifically, the input interface 130 is connected to the processing circuitry 150, and converts an input operation received from the operator into an electrical signal, to output to the processing circuitry 150. For example, the input interface 130 is implemented by a trackball, a switch button, a mouse, a keyboard, a touch pad with which an input operation is made by touching an operating surface, a touch screen in which a display screen and a touch pad are integrated, a non-contact input interface using an optical sensor, a sound input interface, or the like. In the present specification, the input interface 130 is not limited to ones having a physical operating part, such as a mouse and a keyboard. For example, processing circuitry that receives an electrical signal corresponding to an input operation from an external input device that is provided separately from the apparatus, and that outputs this electrical signal to a control circuit is also included in examples of the input interface 130.

The display 140 displays various kinds of information and various kinds of data. Specifically, the display 140 is connected to the processing circuitry 150, and displays various kinds of information and various kinds of data output from the processing circuitry 150. For example, the display 140 is implemented by a liquid crystal monitor, a cathode ray tube (CRT) monitor, a touch panel, or the like.

The processing circuitry 150 controls components of the medical image processing apparatus 100 in response to an input operation received from the operator through the input interface 130. For example, the processing circuitry 150 causes the storage 120 to store a medical image output from the NW interface 110. Moreover, for example, the processing circuitry 150 reads a medical image from the storage 120, and displays it on the display 140.

An overall configuration of the medical image processing apparatus 100 according to the present embodiment has been explained. With the configuration as described, the medical image processing apparatus 100 according to the present embodiment is installed in medical facilities such as a hospital, and is used when diagnosis of an infarction in a subject tissue, such as brain is performed.

For patients with an infarction, because presence of a collateral circulation is an indicator for a possibility of recovery of a tissue, its evaluation is important. The collateral circulation is a circulation vessel that is newly developed, when a stenosis or an occlusion occurs, to compensate a blood flow deficit caused thereby.

Accordingly, a technique of scoring a collateral circulation by using parameters acquired, for example, by perfusion analysis has been proposed. According to this technique, a ratio between an amount of blood flowing into a major artery, such as a cerebral blood volume, and an amount of blood perfused through a brain tissue, such as a cerebral blood volume is calculated for each of a healthy hemisphere and a unhealthy hemisphere (such as a hemisphere having an infarction), and a rate of blood provided by the collateral circulation that flows into a unhealthy tissue can thereby be calculated.

Moreover, it has been known that the collateral circulation includes an anterograde type and a retrograde type. Because the retrograde type is poor in circulation performance compared to the anterograde, it is also important to identify whether the collateral circulation is the anterograde type or the retrograde type.

Therefore, a technique of generating a color map indicating vascular territories of a brain, for example, based on contrast radiography has been proposed. The vascular territories are divided regions of an organ including plural arteries that are divided based on a feeding artery. According to this technique, by dividing a brain into vascular territories based on arrival times of a contrast agent perfused into the brain tissue, not by dividing into analytically defined vascular territories, it is possible to generate an image indicating vascular territories in which presence or absence of an infarction area or a collateral circulation is reflected.

However, both of these techniques are for evaluating a state of a collateral circulation, and it is difficult to quantitatively evaluate a collateral circulation that is directly connected to a treatment decision and a prognosis at the tissue level.

For example, the first technique requires an amount of blood flowing through a major artery to score an amount of collateral circulation, but a time resolution is not sufficient in a medical image diagnostic apparatus (also called modality) that is generally used at diagnosis, and it is difficult to measure an amount of blood flow accurately. Moreover, for example, the second technique visualizes a collateral circulation, but it is difficult to quantitatively grasp which vessel is predominant in a vascular territory having an infarction.

Because of this, the medical image processing apparatus 100 according to the present embodiment is configured to be capable of presenting a quantitative indicator of a collateral circulation that is directly connected to a treatment decision and a prognosis.

Specifically, the processing circuitry 150 includes an acquiring function 151, a generating function 152, a first setting function 153, a second setting function 154, a calculating function 155, and an output function 156. The acquiring function 151 is one example of an acquiring unit. Moreover, the generating function 152 is one example of a generating unit. The first setting function 153 is one example of a first setting unit. The second setting function 154 is one example of a second setting unit. The calculating function 155 is one example of a calculating unit. The output function 156 is one example of an output unit.

The acquiring function 151 acquires medical images of multiple time phases of a subject tissue of a subject to be diagnosed from the medical image diagnostic apparatus 20 or the medical image storage apparatus 30. The generating function 152 generates a vascular territory image that shows plural vascular territories included in the subject tissue of the subject based on the medical images of multiple time phases acquired by the acquiring function 151.

The first setting function 153 sets a region of interest in the subject tissue of the subject. The second setting function 154 sets at least two regions out of the vascular territories and an ischemia area in the region of interest set by the first setting function 153, based on the vascular territory image generated by the generating function 152.

The calculating function 155 calculates a ratio of each of the at least two regions set by the second setting function 154 to the region of interest set by the first setting function 153. The output function 156 outputs information about the ratio calculated by the calculating function 155 to the display 140.

With such a configuration, for vascular territories and an ischemia area included in the region of interest, a quantitative indicator of a collateral circulation that is directly connected to a treatment decision and a prognosis can be presented by outputting information of a ratio of each region and a region of interest.

Hereinafter, respective processing functions of the processing circuitry 150 described above will be explained in detail.

In the following, a case in which a subject tissue to be a subject of diagnosis is brain will be explained as an example. In this case, the first setting function 153 sets a region of interest in a hemisphere having an ischemia area out of two hemispheres of the brain.

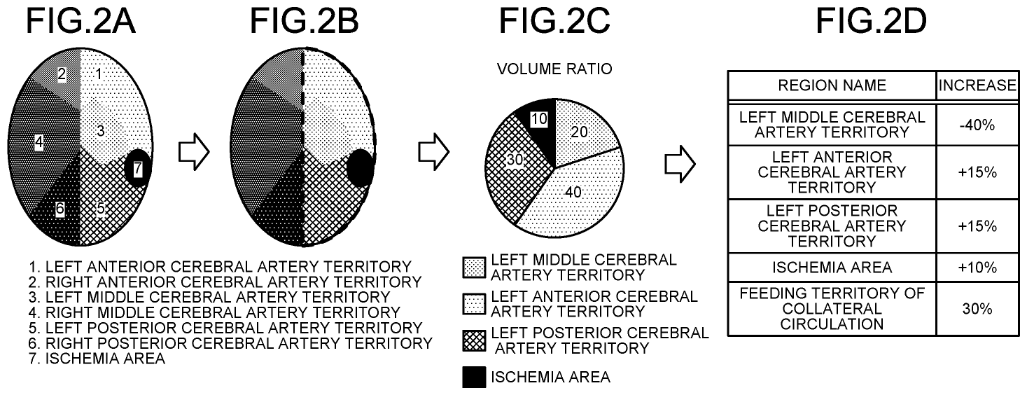

FIGS. 2A, 2B, 2C, and 2D are diagrams for explaining an example of processing performed by the respective processing functions included in the processing circuitry 150 according to the first embodiment.

In the present embodiment, the acquiring function 151 acquires medical images of multiple time phases of a brain of a subject from the medical image diagnostic apparatus 20 or the medical image storage apparatus 30.

The medical image acquired by the acquiring function 151 may be any image as long as it is an image with which an arrival time of a contrast agent can be grasped. For example, the medical image is a CT image acquired by an X-ray CT apparatus, an MRI image acquired by an MRI apparatus, and the like. The medical image may be an image acquired by an imaging method enabling to visualize movement of blood flow without using a contrast agent, such as arteria spin labeling (ASL).

Moreover, the generating function 152 generates a vascular territory image showing a middle cerebral artery territory, an anterior cerebral artery territory, and a posterior cerebral artery territory included in the brain of the subject based on the medical images of multiple time phases acquired by the acquiring function 151.

For example, the generating function 152 divides the brain into regions based on arrival times of a contrast agent perfused into the brain tissue, and thereby generates the vascular territory image showing a vascular territory in which presence or absence of an infarction area or a collateral circulation is reflected. For example, the generating function 152 generates a vascular territory image by a method described in Patent Literature 2.

The vascular territory image generated by the generating function 152 includes, for example, as shown in FIG. 2A, a left anterior cerebral artery territory (region “1”), a left middle cerebral artery territory (region “3”), and a left posterior cerebral artery territory (region “5”) included in a left hemisphere (right side in the drawing), and a right anterior cerebral artery territory (region “2”), a right middle cerebral artery territory (region “4”), and a right posterior cerebral artery territory (region “6”) included in a right hemisphere (left side in the drawing), as vascular territories.

Moreover, the first setting function 153 identifies an entire territory of a cerebral hemisphere on an opposite side to a cerebral hemisphere including an ischemia area by using at least one of the medical images of multiple time phases acquired by the acquiring function 151, and sets an inverted territory of the identified territory relative to a median plane of the brain as a region of interest.

For example, as shown in FIG. 2A, suppose that an ischemia area (region “7”) is included in the left hemisphere. In this case, the first setting function 153 determines that an ischemia area is included in the left hemisphere by using the vascular territory image generated by the generating function 152. The first setting function 153 identifies an entire territory of the right hemisphere that is the opposite hemisphere to the left hemisphere including the ischemia area, by using at least one of the medical images of multiple time phases acquired by the acquiring function 151.

Thereafter, the first setting function 153 estimates an entire territory of the left hemisphere by inverting the right hemisphere in the vascular territory image to the left side relative to the median plane of the brain as shown in FIG. 2B. The first setting function 153 sets the estimated entire territory of the left hemisphere as a region of interest (region surrounded by a broken line in FIG. 2B).

Moreover, the second setting function 154 sets at least two regions out of the middle cerebral artery territory, the anterior cerebral artery territory, the posterior cerebral artery territory, and the ischemia area in the region of interest set by the first setting function 153 based on the vascular territory image generated by the generating function 152.

For example, the second setting function 154 sets, as shown in FIG. 2B, respective regions of the left middle cerebral artery territory, the left anterior cerebral artery territory, the left posterior cerebral artery territory, and the ischemia area in the region of interest.

Moreover, the calculating function 155 calculates a volume ratio of each of the at least two regions to the region of interest, as a ratio of each of the at least two regions set by the second setting function 154 to the region of interest.

For example, the calculating function 155 calculates a volume ratio of each of the at least two regions set by the second setting function 154 to the region of interest as a first volume ratio, calculates a volume ratio of each of the at least two regions included in the hemisphere on the opposite side to the hemisphere including the ischemia area to the entire territory of the hemisphere on the opposite side as a second volume ratio, and calculates a difference between the first volume ratio and the second volume ratio for each corresponding territories.

For example, the calculating function 155 calculates a volume of the right hemisphere corresponding to a volume of the region of interest by using at least one of the medical images of multiple time phases acquired by the acquiring function 151. The calculating function 155 calculates a volume ratio of each of the left middle cerebral artery territory, the left anterior cerebral artery territory, the left posterior cerebral artery territory, and the ischemia area set in the region of interest to the right hemisphere as the first volume ratio.

For example, as shown in FIG. 2C, the calculating function 155 calculates the first volume ratio of the left middle cerebral artery territory, the first volume ratio of the left anterior cerebral artery territory, the first volume ratio of the left posterior cerebral artery territory, and the first volume ratio of the ischemia area as 20%, 40%, 30%, and 10%, respectively, when the volume of the right hemisphere corresponding to the volume of the region of interest is 100%.

Furthermore, the calculating function 155 calculates a volume ratio of each of the right middle cerebral artery territory, the right anterior cerebral artery territory, the right posterior cerebral artery territory, and the ischemia area included in the right hemisphere to the entire territory of the right hemisphere as the second volume ratio by using the vascular image generated by the generating function 152. At this time, because the ischemia area is not included in the right hemisphere, the second volume ratio of the ischemia area is calculated as 0.

The calculating function 155 calculates a difference between the first volume ratio and the second volume ratio for each left and right corresponding regions. At this time, for example, the calculating function 155 calculates an increase in volume ratio from the second volume ratio to the first volume ratio as the difference between the first volume ratio and the second volume ratio by subtracting the second volume ratio from the first volume ratio. Furthermore, regarding a vascular territory in which the volume ratio has increased as a feeding territory of a collateral circulation, the calculating function 155 calculates a sum of the volume ratios of the vascular territory as an increase in volume ratio of the feeding territory of the collateral circulation.

For example, as shown in FIG. 2D, the calculating function 155 calculates an increase in volume ratio of the left middle cerebral artery territory, an increase in volume ratio of the left anterior cerebral artery territory, an increase in volume ratio of the left posterior cerebral artery territory, an increase in volume ratio of the ischemia area, and an increase in volume ratio of the feeding territory of the collateral circulation as −40%, +15%, +15%, +10%, and 30%, respectively.

Moreover, the output function 156 outputs information indicating the first volume ratio, and the difference between the first volume ratio and the second volume ratio calculated by the calculating function 155 for each of the at least two regions set by the second setting function 154 to the display 140.

For example, as shown in FIG. 2C, the output function 156 outputs information indicating the first volume ratio calculated by the calculating function 155 for each of the left middle cerebral artery territory, the left anterior cerebral artery territory, the left posterior cerebral artery territory, and the ischemia area. For example, the output function 156 outputs information indicating the first volume ratios of the respective regions in a form of pie chart.

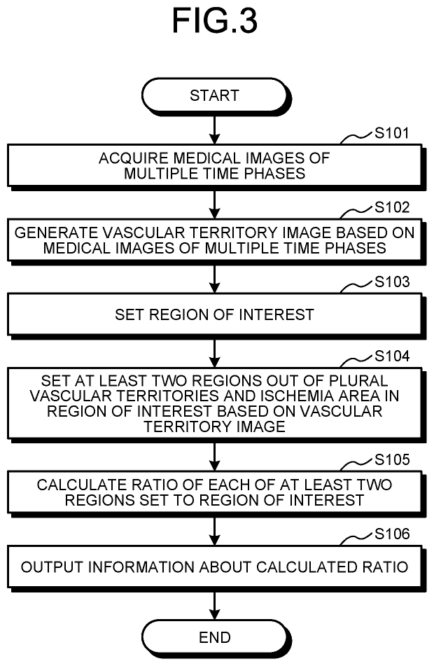

Moreover, for example, as shown in FIG. 2D, the output function 156 outputs information indicating a difference between the first volume ratio and the second volume ratio calculated by the calculating function 155 for each of the left middle cerebral artery territory, the left anterior cerebral artery territory, the left posterior cerebral artery territory, and the ischemia area. For example, the output function 156 outputs information indicating increases in volume ratio of the respective regions in a form of list shown on the left side of FIG. 3.

The respective processing functions of the processing circuitry 150 has so far been explained. For example, the processing circuitry 150 is implemented by a processor. In this case, the respective processing functions described above are stored in the storage 120 in a form of computer-executable programs. The processing circuitry 150 implements functions corresponding to the respective programs by reading and executing the respective programs stored in the storage 120. In other words, the processing circuitry 150 is to have the respective functions illustrated in FIG. 1 in a state in which the processing circuitry 150 has read the respective programs.

FIG. 3 is a flowchart illustrating a procedure of processing performed by the processing circuitry 150 according to the first embodiment.

For example, as illustrated in FIG. 3, the processing circuitry 150 first acquires medical images of multiple time phases of a subject tissue of a subject from the medical image diagnostic apparatus 20 or the medical image storage apparatus 30 (step S101). This step is a step corresponding to the acquiring function 151. For example, the processing circuitry 150 performs this step by reading and executing a program corresponding to the acquiring function 151 from the storage 120.

Subsequently, the processing circuitry 150 generates a vascular territory image showing plural vascular territories included in the subject tissue of the subject based on the medical images of multiple time phases (step S102). This step is a step corresponding to the generating function 152. For example, the processing circuitry 150 performs this step by reading and executing a program corresponding to the generating function 152 from the storage 120.

Subsequently, the processing circuitry 150 sets a region of interest in the subject tissue of the subject (step S103). This step is a step corresponding to the first setting function 153. For example, the processing circuitry 150 performs this step by reading and executing a program corresponding to the first setting function 153 from the storage 120.

Subsequently, the processing circuitry 150 sets at least two regions out of the vascular territories and an ischemia area in the region of interest based on the vascular territory image (step S104). This step is a step corresponding to the second setting function 154. For example, the processing circuitry 150 performs this step by reading and executing a program corresponding to the second setting function 154 from the storage 120.

Subsequently, the processing circuitry 150 calculates a ratio of each of the set at least two regions and the region of interest (step S105). This step is a step corresponding to the calculating function 155. For example, the processing circuitry 150 performs this step by reading and executing a program corresponding to the calculating function 155 from the storage 120.

Subsequently, the processing circuitry 150 outputs information about the calculated ratio to the display 140 (step S106). This step is a step corresponding to the output function 156. For example, the processing circuitry 150 performs this step by reading and executing a program corresponding to the output function 156 from the storage 120.

As described above, in the first embodiment, the acquiring function 151 acquires medical images of multiple time phases of a subject tissue of a subject. Moreover, the generating function 152 generates a vascular territory image showing plural vascular territories included in the subject tissue of the subject based on the medical images of multiple time phases. Moreover, the first setting function 153 sets a region of interest. Furthermore, the second setting function 154 sets at least two regions out of the vascular territories and an ischemia area in the region of interest based on the vascular territory image. Moreover, the calculating function 155 calculates a ratio of each of the at least two regions to the region of interest set by the first setting function 153. The output function 156 outputs information about the ratio.

With such a configuration, for vascular territories and an ischemia area included in a region of interest, a quantitative indicator of a collateral circulation that is directly connected to a treatment decision and a prognosis can be presented by outputting information about a ratio of each region to a region of interest. Moreover, a blood vessel that is predominant in the region of interest can be quantitatively grasped.

Moreover, in the first embodiment, the calculating function 155 calculates a volume ratio of each of the at least two regions to a region of interest, as a ratio of each of the at least two regions and the region of interest. With such a configuration, information about the volume ratio of each of the vascular territories or an ischemia area set in the region of interest to the region of interest can be presented as a quantitative indicator of a collateral circulation. Moreover, a blood vessel that is predominant in the region of interest can be grasped more accurately.

Moreover, in the first embodiment, a subject tissue to be diagnosed is brain. The first setting function 153 sets a region of interest in a hemisphere including an ischemia area out of two hemispheres of the brain. With such a configuration, a quantitative indicator of a collateral circulation in a brain can be presented.

Moreover, in the first embodiment, the first setting function 153 identifies an entire territory of a hemisphere on the opposite side to a hemisphere including an ischemia area by using at least one of medical images of multiple time phases, and sets an inverted territory of the identified territory relative to a median plane of the brain as a region of interest. With such a configuration, information about a ratio between the entire territory of the hemisphere and each vascular territory or the ischemia area can be presented as a quantitative indicator of a collateral circulation.

Moreover, in the first embodiment, the calculating function 155 calculates a volume ratio of each of at least two regions to a region of interest as a first volume ratio, calculates a volume ratio of the at least two regions included in a hemisphere on the opposite side to a hemisphere including an ischemia area to an entire territory of the hemisphere on the opposite side as a second volume ratio, and calculates a difference between the first volume ratio and the second volume ratio for each corresponding territories. With such a configuration, an increase and decrease in size of respective vascular territories and the ischemia area can be easily grasped.

As above, the first embodiment has been explained, but the medical image processing apparatus 100 can also be implemented by changing a part of the configuration appropriately. In the following, a modification of the first embodiment will be explained as another embodiment. In the following embodiment, what is different from the first embodiment will be mainly explained, and detailed explanation of duplicated part will be omitted.

### Second Embodiment

For example, in the first embodiment described above, a case in which an entire territory of a hemisphere on the opposite side to a hemisphere including an ischemia area is identified by using at least one of medical images of multiple time phases, and a region of interest is set based on the territory has been explained, but embodiments are not limited thereto.

For example, by using a vascular territory image, a vascular territory including a position corresponding to a position of an ischemia area in a hemisphere on the opposite side to a hemisphere including the ischemia area may be identified, and a region of interest may be set based on the vascular territory. In the following, such an example will be explained as a second embodiment.

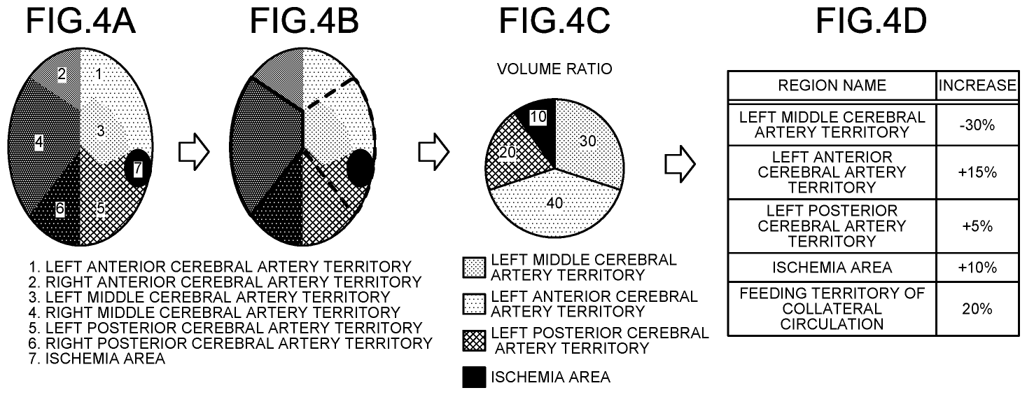

FIGS. 4A, 4B, 4C, and 4D are diagrams for explaining an example of processing performed by respective processing functions included in the processing circuitry 150 according to the second embodiment.

In the present embodiment, the first setting function 153 detects a position of an ischemia area in a hemisphere including the ischemia area by using a vascular territory image generated by the generating function 152, identifies a vascular territory including a position corresponding to the position of the ischemia area in a hemisphere on the opposite side to the hemisphere including the ischemia area, and sets an inverted territory of the identified vascular territory relative to a median plane of the brain as a region of interest.

For example, as shown in FIG. 4A, suppose that an ischemia area (region “7”) is included in a left hemisphere. In this case, the first setting function 153 determines that an ischemia area is included in the left hemisphere by using a vascular territory image, and detects a position of the ischemia area. The first setting function 153 further identifies a vascular territory including a position corresponding to the position of the ischemia area in a right hemisphere that is the opposite side to the left hemisphere including the ischemia area by using the vascular territory image.

Suppose, for example, a vascular territory including a position corresponding to the ischemia area in the right hemisphere is the right middle cerebral artery territory (region “4”). In this case, the first setting function 153 inverts the right middle cerebral artery territory in the vascular territory image to the left side relative to the median plane of the brain as shown in FIG. 4B, and sets the inverted territory as the region of interest (territory surrounded by a broken line shown in FIG. 4B).

Moreover, the second setting function 154 sets at least two regions out of middle cerebral artery territory, the anterior cerebral artery territory, the posterior cerebral artery territory, and the ischemia area in the region of interest set by the first setting function 153, similarly to the first embodiment although the region of interest is different.

For example, the second setting function 154 sets the left middle cerebral artery territory, the left anterior cerebral artery territory, the left posterior cerebral artery territory, and the ischemia area in the region of interest as shown in FIG. 4B.

Moreover, the calculating function 155 calculates a volume ratio of each of the at least two regions set by the second setting function 154 to the region of interest, similarly to the first embodiment although the region of interest is different.

For example, the calculating function 155 calculates a volume of the right middle cerebral artery territory corresponding to the volume of the region of interest by using the vascular territory image generated by the generating function 152. The calculating function 155 calculates a volume ratio of each of the left middle cerebral artery territory, the left anterior cerebral artery territory, the left posterior cerebral artery territory, and the ischemia area to the right middle cerebral artery territory as the first volume ratio.

For example, as shown in FIG. 4C, the calculating function 155 calculates the first volume ratio of the left middle cerebral artery territory, the first volume ratio of the left anterior cerebral artery territory, the first volume ratio of the left posterior cerebral artery territory, and the first volume ratio the ischemia area as 30%, 40%, 20%, and 10%, respectively when the volume of the right middle cerebral artery territory corresponding to the volume of the region of interest is 100%.

Furthermore, the calculating function 155 calculates a volume ratio of each of the right middle cerebral artery territory, the right anterior cerebral artery territory, the right posterior cerebral artery territory, and the ischemia area included in the right hemisphere to the entire territory of the right hemisphere as the second volume ratio similarly to the first embodiment. The calculating function 155 calculates a difference between the first volume ratio and the second volume ratio for each left and right corresponding regions similarly to the first embodiment.

For example, as shown in FIG. 4D, the calculating function 155 calculates an increase in volume ratio of the left middle cerebral artery territory, an increase in volume ratio of the left anterior cerebral artery territory, an increase in volume ratio of the left posterior cerebral artery territory, an increase in volume ratio of the ischemia area, an increase in volume ratio of a feeding territory of a collateral circulation as −30%, +15%, +5%, +10%, and 20%, respectively.

Moreover, the output function 156 outputs information indicating the first volume ratio, and the difference between the first volume ratio and the second volume ratio calculated by the calculating function 155 for each of the at least two regions set by the second setting function 154 to the display 140, similarly to the first embodiment.

For example, as shown in FIG. 4C, the output function 156 outputs information indicating the first volume ratio calculated by the calculating function 155 for each of the left middle cerebral artery territory, the left anterior cerebral artery territory, the left posterior cerebral artery territory, and the ischemia area. Moreover, for example, as shown in FIG. 4D, the output function 156 outputs information indicating a difference between the first volume ratio and the second volume ratio calculated by the calculating function 155 for each of the left middle cerebral artery territory, the left anterior cerebral artery territory, the left posterior cerebral artery territory, and the ischemia area.

As described above, in the second embodiment, the first setting function 153 detects a position of an ischemia area in a hemisphere including the ischemia area by using a vascular territory image, identifies a vascular territory including a position corresponding to the position of the ischemia area in a hemisphere on the opposite side to the hemisphere including the ischemia area, and sets an inverted territory of the identified territory relative to a median plane of the brain as a region of interest. With such a configuration, information about a ratio between a specific vascular territory and each vascular territory or an ischemia area as a quantitative indicator of a collateral circulation.

### Third Embodiment

Moreover, for example, in the second embodiment described above, a case in which a vascular territory including a position corresponding to a position of an ischemia area in a hemisphere on the opposite side to a hemisphere including the ischemia area is identified by using a vascular territory image, and a region of interest is set based on the vascular territory has been explained, but embodiments are not limited thereto.

For example, a vascular territory including a position of an ischemia area detected by a vascular territory image may be identified by using a healthy vascular-territory image showing a vascular territory in a healthy condition, and a region of interest may be set based on the vascular territory. In the following, such an example will be explained as a third embodiment.

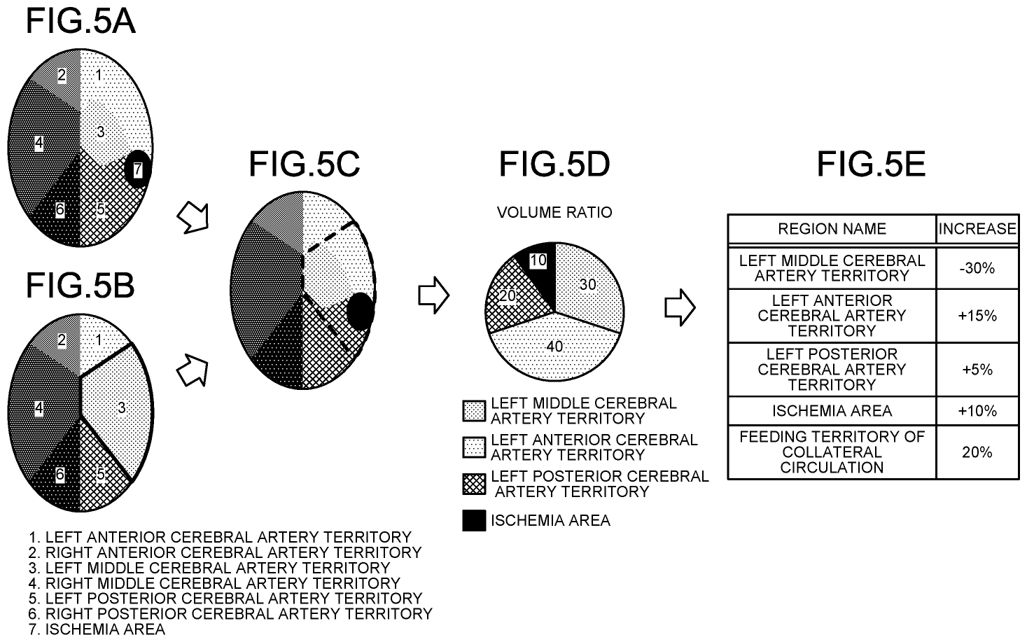

FIGS. 5A, 5B, 5C, 5D, and 5E are diagrams for explaining an example of processing performed by respective processing functions included in the processing circuitry 150 according to the third embodiment.

In the present embodiment, the acquiring function 151 further acquires a healthy vascular-territory image showing a vascular territory in a healthy condition from the medical image diagnostic apparatus 20 or the medical image storage apparatus 30.

For example, the acquiring function 151 acquires an image showing anatomically defined vascular territories, such as an atlas image, a vascular territory image after treatment of a subject to be diagnosed, and the like as a healthy vascular-territory image.

Moreover, the first setting function 153 detects a position of an ischemia area in a hemisphere including the ischemia area by using the vascular territory image generated by the generating function 152, identifies a vascular territory including a position of the ischemia area detected in the vascular territory image by using the healthy vascular-territory image acquired by the acquiring function 151, and sets a region in which the identified vascular territory is mapped in the vascular territory image as a region of interest.

For example, as shown in FIG. 5A, suppose that an ischemia area (region “7”) is included in a left hemisphere. In this case, the first setting function 153 determines that an ischemia area is included in a left hemisphere by using the vascular territory image generated by the generating function 152. The first setting function 153 identifies a vascular territory including a position of an ischemia area detected in the vascular territory image by using the healthy vascular-territory image acquired by the acquiring function 151.

For example, as shown in FIG. 5B, suppose that a vascular territory including the position of the ischemia area in the healthy vascular-territory image is the left middle cerebral artery territory (region “3”). In this case, the first setting function 153 estimates the left middle cerebral artery territory in a healthy condition in the hemisphere including the ischemia area by mapping the left middle cerebral artery territory in the healthy vascular-territory image in the vascular territory image. The first setting function 153 sets the estimated vascular territory as a region of interest (region surrounded by a broken line shown in FIG. 5C).

Moreover, the second setting function 154 sets at least two regions out of the middle cerebral artery territory, the anterior cerebral artery territory, the posterior cerebral artery territory, and the ischemia area in the region of interest set by the first setting function 153.

For example, the second setting function 154 respectively sets the left middle cerebral artery territory, the left anterior cerebral artery territory, the left posterior cerebral artery territory, and the ischemia area in the region of interest as shown in FIG. 5C.

Moreover, the calculating function 155 calculates a volume ratio of each of the at least two regions set by the second setting function 154 to the region of interest, similarly to the first embodiment although the region of interest is different.

For example, the calculating function 155 calculates a volume of the left middle cerebral artery territory corresponding to a volume of the region of interest by using the healthy vascular-territory image acquired by the acquiring function 151. The calculating function 155 calculates a volume ratio of each of the left middle cerebral artery territory, the left anterior cerebral artery territory, the left posterior cerebral artery territory, and the ischemia area set in the region of interest to the left middle cerebral artery territory as a first volume ratio.

For example, as shown in FIG. 5D, the calculating function 155 calculates the first volume ratio of the left middle cerebral artery territory, the first volume ratio of the left anterior cerebral artery territory, the first volume ratio of the left posterior cerebral artery territory, and the first volume ratio of the ischemia area as 30%, 40%, 20%, and 10%, respectively, when the volume of the left middle cerebral artery territory corresponding to the volume of the region of interest is 100%.

Furthermore, the calculating function 155 calculates a volume ratio of each of the right middle cerebral artery territory, the right anterior cerebral artery territory, the right posterior cerebral artery territory, and the ischemia area included in the right hemisphere to the entire territory of the right hemisphere as the second volume ratio, similarly to the first embodiment. The calculating function 155 calculates a difference between the first volume ratio and the second volume ratio for each left and right corresponding regions, similarly to the first embodiment.

For example, as shown in FIG. 5E, the calculating function 155 calculates an increase in volume ratio of the left middle cerebral artery territory, an increase in volume ratio of the left anterior cerebral artery territory, an increase in volume ratio of the left posterior cerebral artery territory, an increase in volume ratio of the ischemia area, and an increase in volume ratio of the feeding territory of the collateral circulation as −30%, +15%, +5%, +10%, and 20%, respectively.

Moreover, the output function 156 outputs information indicating the first volume ratio calculated by the calculating function 155, and a difference between the first volume ratio and the second volume ratio of the at least two regions set by the second setting function 154 to the display 140, similarly to the first embodiment.

For example, as shown in FIG. 5D, the output function 156 outputs information indicating the first volume ratio calculated by the calculating function 155 for each of the left middle cerebral artery territory, the left anterior cerebral artery territory, the left posterior cerebral artery territory, and the ischemia area. Moreover, for example, as shown in FIG. 5E, the output function 156 outputs information indicating a difference between the first volume ratio and the second volume ratio calculated by the calculating function 155 for each of the left middle cerebral artery territory, the left anterior cerebral artery territory, the left posterior cerebral artery territory, and the ischemia area.

As described above, in the third embodiment, the acquiring function 151 further acquires a healthy vascular-territory image showing a vascular territories in a healthy condition. The first setting function 153 detects a position of an ischemia area in a hemisphere including the ischemia area by using the vascular territory image, identifies a vascular territory including the position of the ischemia area detected in the vascular territory image by using the healthy vascular-territory image, and sets a region in which the identified vascular territory is mapped in the vascular territory image as a region of interest. With such a configuration, information about a ratio between a vascular territory in a healthy condition and each vascular territory or an ischemia area can be presented as a quantitative indicator of a collateral circulation.

### Fourth Embodiment

Moreover, for example, in the third embodiment, a case in which a vascular territory including a position of an ischemia area detected in a vascular territory image is identified by using a healthy vascular-territory image showing vascular territories in a healthy condition, and a region of interest is set based on the vascular territory has been explained, but embodiments are not limited thereto.

For example, it may be configured to identify a low perfusion area in a hemisphere by using a perfusion image of the same subject, and set a region of interest based on the low perfusion area. In the following, such an example will be explained as a fourth embodiment.

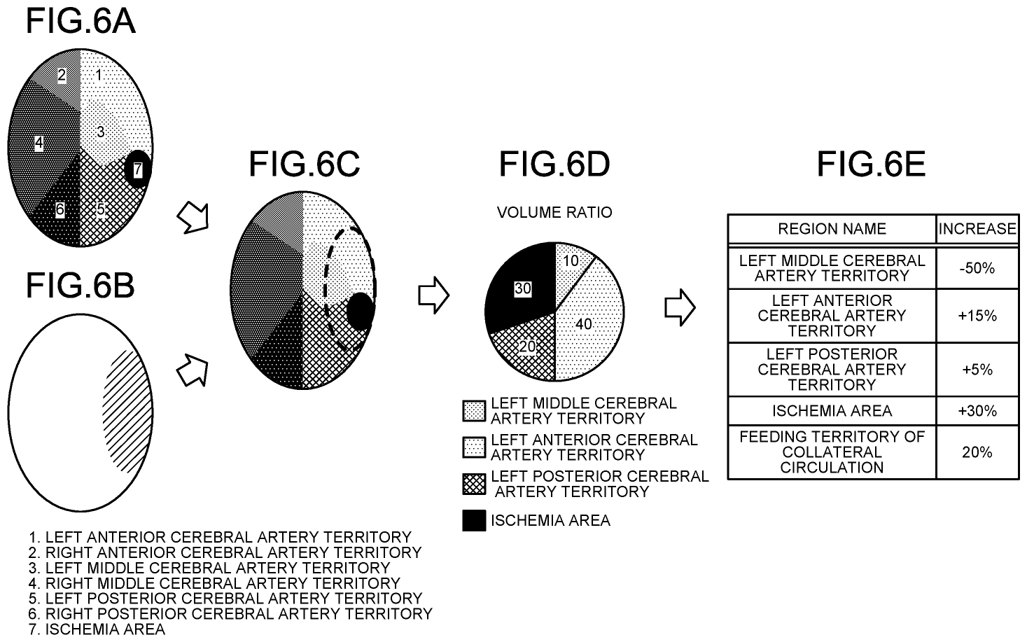

FIGS. 6A, 6B, 6C, 6D, and 6E are diagrams for explaining an example of processing performed by respective processing functions included in the processing circuitry 150 according to the fourth embodiment.

In the present embodiment, the acquiring function 151 further acquires a perfusion image of a subject to be diagnosed from the medical image diagnostic apparatus 20 or the medical image storage apparatus 30.

Moreover, the first setting function 153 identifies a low perfusion area in a hemisphere including an ischemia area by using the perfusion image acquired by the acquiring function 151, and sets a region in which the identified low perfusion area is mapped in a vascular territory image as a region of interest.

At this time, for example, the first setting function 153 identifies the low perfusion area based on perfusion parameters acquired from the perfusion image. For example, the first setting function 153 identifies a region in which Tmax exceeds 6 s or a region in which a contralateral ratio cerebral blood flow (CBF) is lower than 30% as the low perfusion area.

For example, as shown in FIG. 6A, suppose that an ischemia area (region “7”) is included in a left hemisphere. In this case, the first setting function 153 determines that an ischemia area is included in the left hemisphere by using the vascular territory image generated by the generating function 152, and detects a position of the ischemia area. The first setting function 153 identifies a low perfusion area (diagonally shaded region shown in FIG. 6B) in the hemisphere including the ischemia area by using the perfusion image acquired by the acquiring function 151 as shown in FIG. 6B.

Thereafter, the first setting function 153 maps the perfusion area in the perfusion image into the vascular territory image as shown in FIG. 6C, and sets the mapped region as a region of interest (region surrounded by a broken line in FIG. 6C).

Moreover, the second setting function 154 sets at least two regions out of the middle cerebral artery territory, the anterior cerebral artery territory, the posterior cerebral artery territory, and the ischemia area in the region of interest set by the first setting function 153, similarly to the first embodiment although the region of interest is different.

For example, the second setting function 154 sets the left middle cerebral artery territory, the left anterior cerebral artery territory, the left posterior cerebral artery territory, and the ischemia area in the region of interest as shown in FIG. 6C.

Moreover, the calculating function 155 calculates a volume ratio of each of the at least two regions set by the second setting function 154 to the region of interest, similarly to the first embodiment although the region of interest is different.

For example, the calculating function 155 calculates a volume of a low perfusion area corresponding to the volume of the region of interest by using the perfusion image acquired by the acquiring function 151. The calculating function 155 calculates a volume ratio of each of the left middle cerebral artery territory, the left anterior cerebral artery territory, the left posterior cerebral artery territory, and the ischemia area included in the region of interest to the right middle cerebral artery territory as the first volume ratio.

For example, as shown in FIG. 6D, the calculating function 155 calculates the first volume ratio of the left middle cerebral artery territory, the first volume ratio of the left anterior cerebral artery territory, the first volume ratio of the left posterior cerebral artery territory, and the first volume ratio of the ischemia area as 10%, 40%, 20%, and 30%, respectively, when the volume of the low perfusion area corresponding to the volume of the region of interest is 100%.

Furthermore, the calculating function 155 calculates a volume ratio of each of the right middle cerebral artery territory, the right anterior cerebral artery territory, the right posterior cerebral artery territory, and the ischemia area included in the right hemisphere to the entire territory of the right hemisphere as the second volume ratio, similarly to the first embodiment. The calculating function 155 calculates a difference between the first volume ratio and the second volume ratio for each left and right corresponding regions similarly to the first embodiment.

For example, as shown in FIG. 6E, the calculating function 155 calculates an increase in volume ratio of the left middle cerebral artery territory, an increase in volume ratio of the left anterior cerebral artery territory, an increase in volume ratio of the left posterior cerebral artery territory, an increase in volume ratio of the ischemia area, and an increase in volume ratio of the feeding territory of the collateral circulation as −50%, +15%, +5%, +30%, and 20%, respectively.

Moreover, the output function 156 outputs information indicating the first volume ratio calculated by the calculating function 155, and the difference between the first volume ratio and the second volume ratio of each of the at least two regions set by the second setting function 154 to the display 140, similarly to the first embodiment.

For example, as shown in FIG. 6D, the output function 156 outputs information indicating the first volume calculated by the calculating function 155 for each of the left middle cerebral artery territory, the left anterior cerebral artery territory, the left posterior cerebral artery territory, and the ischemia area. Moreover, for example, as shown in FIG. 6E, the output function 156 outputs information indicating the difference between the first volume ratio and the second volume ratio calculated by the calculating function 155 for each of the left middle cerebral artery territory, the left anterior cerebral artery territory, the left posterior cerebral artery territory, and the ischemia area.

As described above, in the fourth embodiment, the acquiring function 151 further acquires a perfusion image of a subject. The first setting function 153 identifies a low perfusion area in a hemisphere including an ischemia area by using the perfusion image, and sets a region in which the identified low perfusion area is mapped in the vascular territory image as a region of interest. With such a configuration, information about a ratio between a low perfusion area and each vascular territory or an ischemia area can be presented as a quantitative indicator of a collateral circulation.

### Fifth Embodiment

Moreover, for example, in the first to the fourth embodiments, a case in which a ratio between each region and a region of interest is calculated per region for plural vascular territories and an ischemia area has been explained, but embodiments are not limited thereto.

For example, respective regions may be classified into plural blood flow patterns including an anterograde perfusion area, a retrograde perfusion area, and an ischemia area, and a ratio between each region and a region of interest may be calculated for each blood flow pattern. In the following, such an example will be explained as a fifth embodiment.

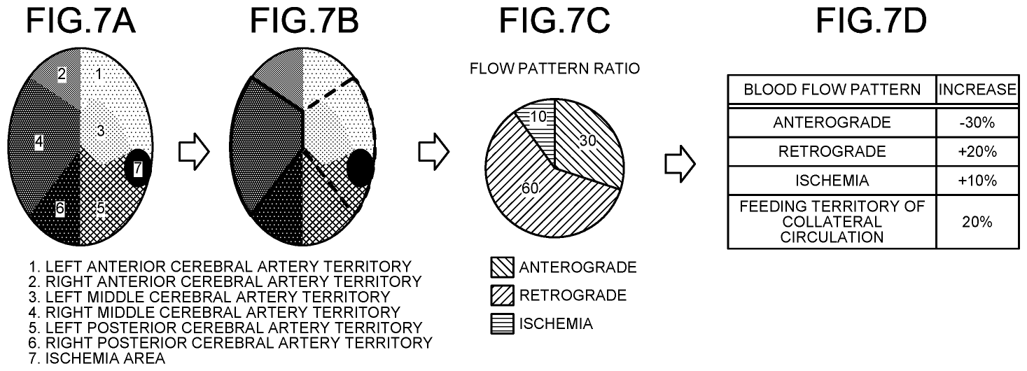

FIGS. 7A, 7B, 7C, and 7D are diagrams for explaining an example of processing performed by respective processing functions included in the processing circuitry 150 according to the fifth embodiment.

In the present embodiment, the calculating function 155 classifies the at least two regions set by the second setting function 154 into plural blood flow patterns including a anterograde perfusion area, a retrograde perfusion area, and an ischemia area, and calculates a ratio between each region and a region of interest for each blood flow pattern.

The anterograde perfusion area is an area in which there is no difference in a fed artery from a healthy condition. The retrograde perfusion area is an area in which a fed artery differs from a state in a healthy condition. Moreover, the ischemia area is an area in which an arrival time of a contrast agent is predetermined time or longer, and in which perfusion is not observed for the predetermined time.

Specifically, after calculating an increase of each of the vascular territories similarly to either one of the first to the fourth embodiments, the calculating function 155 classifies a vascular territory in which the volume ratio has decreased as the anterograde perfusion area, and classifies a vascular territory in which the volume ratio has increased as the retrograde perfusion area. Moreover, the calculating function 155 classifies an ischemia area as the ischemia area.

For example, as shown in the example in FIGS. 4A-4D, suppose that an increase in volume ratio of the left middle cerebral artery territory, an increase in volume ratio of the left anterior cerebral artery territory, and an increase in volume ratio of the left posterior cerebral artery territory are −30%, +15%, and +5%, respectively. In this case, the calculating function 155 classifies the left middle cerebral artery territory as the anterograde perfusion area, classifies the left anterior cerebral artery territory and the left posterior cerebral artery territory as the retrograde perfusion area, and classifies the ischemia area as the ischemia area.

The calculating function 155 calculates a first volume ratio that is a volume ratio to the region of interest, for each blood pattern by summing up the first volume ratios for vascular territories classified into the same blood pattern.

For example, as shown in the example in FIGS. 4A-4D, suppose that the first volume ratio of the left middle cerebral artery territory, the first volume ratio of the left anterior cerebral artery territory, the first volume ratio of the left posterior cerebral artery territory, and the first volume ratio of the ischemia area are 30%, 40%, 20%, and 10%, respectively. In this case, as shown in FIG. 7C, the calculating function 155 calculates the first volume ratio of the anterograde perfusion area (Anterograde), the first volume ratio of the retrograde perfusion area (Retrograde), and the first volume ratio of the ischemia area (Ischemia) are 30%, 60%, and 10%, respectively, when the volume of the region of interest is 100%.

Furthermore, the calculating function 155 calculates a difference between the first volume ratio and the second volume ratio for each blood flow pattern by summing up differences between the first volume ratio and the second volume ratio of vascular territories classified into the same blood flow pattern. At this time, for example, the calculating function 155 calculates an increase in volume ratio from the second volume ratio to the first volume ratio as the difference between the first volume ratio and the second volume ratio for each blood flow pattern.

For example, as shown in FIG. 7D, the calculating function 155 calculates an increase in volume ratio of the anterograde perfusion area (Anterograde), an increase in volume ratio of the retrograde perfusion area (Retrograde), an increase in volume ratio of the ischemia area (Ischemia), and an increase in volume ratio of the feeding territory of a collateral circulation as −30%, +20%, +10%, and 20%, respectively.

Moreover, the output function 156 outputs information indicating the first volume ratio calculated by the calculating function 155, and the difference between the first volume ratio and the second volume ratio to the display 140 for each blood flow pattern.

For example, as shown in FIG. 7C, the output function 156 outputs information indicating the first volume ratio calculated by the calculating function 155 for each of the anterograde perfusion area (Anterograde), the retrograde perfusion area (Retrograde), and the ischemia area (Ischemia). Moreover, for example, as shown in FIG. 7D, the output function 156 outputs information indicating the difference between the first volume ratio and the second volume ratio calculated by the calculating function 155 for each of the anterograde perfusion area (Anterograde), the retrograde perfusion area (Retrograde), and the ischemia area (Ischemia).

As described above, in the fifth embodiment, the calculating function 155 classifies at least two regions into plural blood patterns including the anterograde perfusion area, the retrograde perfusion area, and the ischemia area, and calculates a ratio of each region to a region of interest for each of the blood flow patterns. With such a configuration, information about a ratio between a region of interest and each of vascular territories or an ischemia area can be presented as a quantitative indicator of a collateral circulation for each blood flow pattern.

### Sixth Embodiment

Moreover, for example, in the fifth embodiment described above, a case in which information about a ratio calculated for each blood flow pattern is output has been explained as an example, but embodiments are not limited thereto.

For example, a vascular territory image showing at least one range out of plural blood flow patterns may be further output. In the following, such an example will be explained as a sixth embodiment.

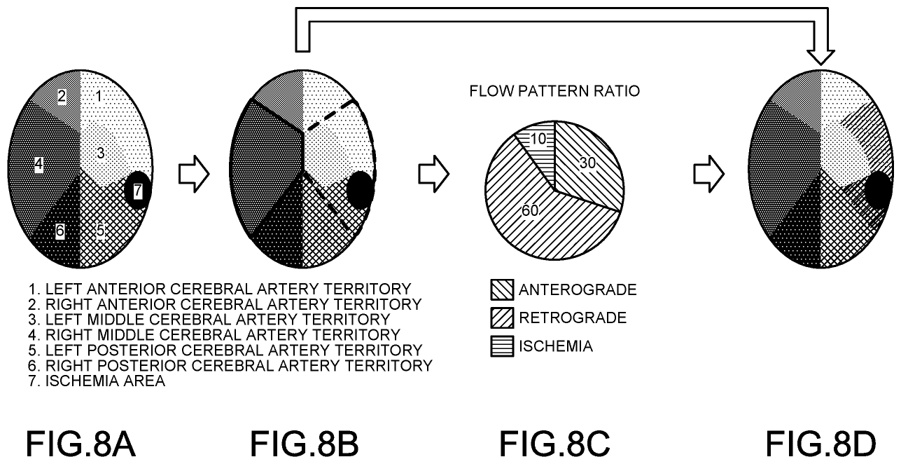

FIGS. 8A, 8B, 8C, and 8D are diagrams for explaining an example of processing performed by respective processing functions included in the processing circuitry 150 according to the sixth embodiment.

In the present embodiment, the calculating function 155 classifies at least two regions set by the second setting function 154 into plural blood flow patterns including an anterograde perfusion area, a retrograde perfusion area, and an ischemia area, similarly to the fifth embodiment.

Moreover, the output function 156 further outputs a vascular territory image showing a region of at least one blood flow pattern out of the plural blood flow patterns classified by the calculating function 155 to the display 140.

For example, as shown in FIG. 8D, the output function 156 outputs a vascular territory image showing the left anterior cerebral artery territory and the left posterior cerebral artery territory that have been classified into the retrograde perfusion area (Retrograde) in a display mode different from that of the other blood flow patterns.

Alternatively, the output function 156 may display the vascular territory classified into the anterograde perfusion area (Anterograde) in a different display mode.

As for a method of varying the display modes of the blood flow patterns, any method may be used as long as it is a method enabling to distinguish from other regions. For example, colors may be varied, or patterns, textures, or the like may be varied.

As described above, in the sixth embodiment, the output function 156 further outputs a vascular territory image showing a region of at least one blood flow pattern out of plural blood flow patterns. With such a configuration, a region of a specific blood flow pattern can be easily grasped on the vascular territory image.

### Seventh Embodiment

Moreover, for example, in the first to the sixth embodiments, a case in which a ratio between each region and a region of interest is calculated for plural vascular territories and an ischemia area has been explained, but embodiments are not limited thereto.

For example, it may be configured to determine a treatment to be adopted to a subject tissue based on a ratio between each region and a region of interest. In the following, such an example will be explained as a seventh embodiment.

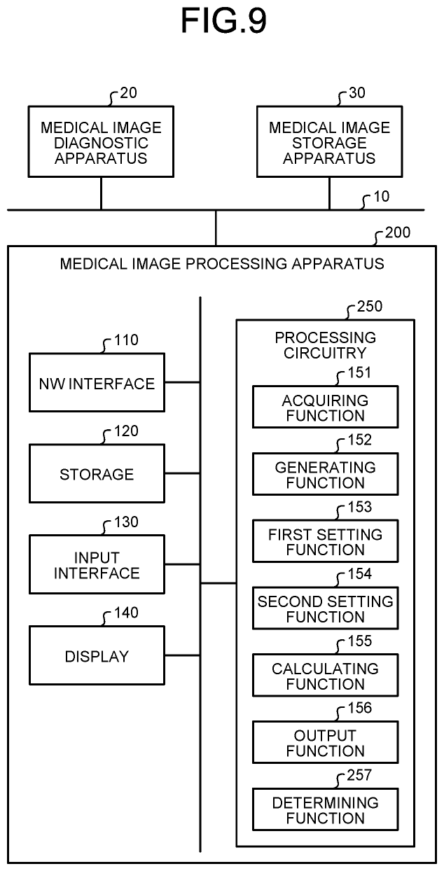

FIG. 9 is a diagram illustrating an example of a configuration of a medical image processing apparatus according to the seventh embodiment.

For example, as illustrated in FIG. 9, in a medical image processing apparatus 200 according to the present embodiment, processing circuitry 250 further includes a determining function 257 in addition to the acquiring function 151, the generating function 152, the first setting function 153, the second setting function 154, the calculating function 155, and the output function 156 illustrated in FIG. 1. The determining function 257 is one example of a determining unit.

The determining function 257 determines a treatment to be adopted to a subject tissue of a subject based on a ratio between each of at least two regions and a region of interest calculated by the calculating function 155.

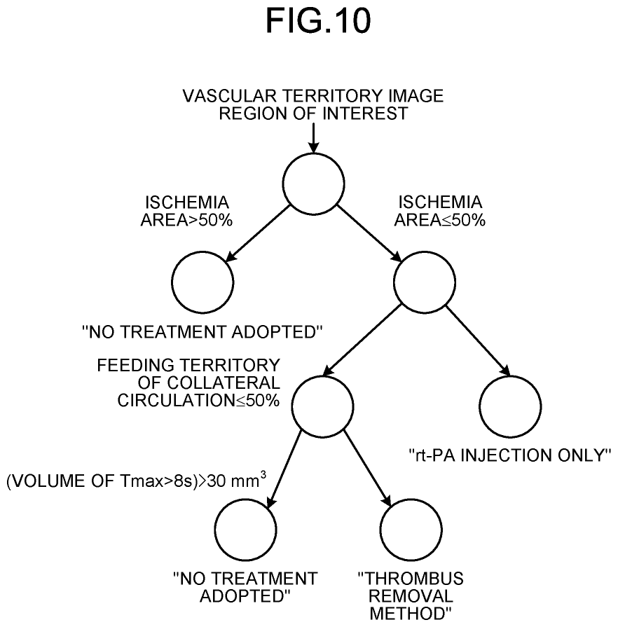

FIG. 10 is a diagram for explaining an example of processing performed by the determining function 257 included in the processing circuitry 250 according to the seventh embodiment.

For example, as illustrated in FIG. 10, the determining function 257 determines a treatment to be adopted to the brain of a subject by using a decision tree having treatment methods in leaf nodes. In this example, similarly to the fourth embodiment, it is assumed that a region of interest is set based on a low perfusion area identified by using a perfusion image, and an increase in volume ratio has been calculated for each region set in the region of interest.

For example, the determining function 257 first determines whether an increase in volume ratio of an ischemia area (Ischemia) calculated by the calculating function 155 is higher than 50%. When the increase in volume ratio of the ischemia area (Ischemia) is higher than 50%, the determining function 257 determines a treatment method as “no treatment adopted”.

On the other hand, when the increase in volume ratio of the ischemia area (Ischemia) is lower than 50%, the determining function 257 determines whether an increase in volume ratio of a feeding territory of a collateral circulation calculated by the calculating function is higher than 50%. When the increase in volume ratio of the feeding territory of a collateral circulation is higher than 50%, the determining function 257 determines the treatment method as “rt-PA injection only”.

On the other hand, when the increase in volume ratio of the feeding territory of a collateral circulation is lower than 50%, the determining function 257 determines whether a volume of a region in which Tmax is larger than 8 s out of low perfusion areas in a perfusion image acquired by the acquiring function 151 is larger than 30 mm³. When the volume of the region in which Tmax is larger than 8 s is larger than 30 mm³, the determining function 257 determines the treatment method as “no treatment adopted”. On the other hand, when the volume of the region in which Tmax is larger than 8 s is smaller than 30 mm³, the determining function 257 determines the treatment method as the “thrombus removal method”.

A case in which the determining function 257 uses an increase in volume ratio of an ischemia area has been explained herein as an example, but an indicator value used for determination of a treatment to be adopted is not limited thereto. For example, an increase in volume ratio of a vascular territory may be used, or an increase in volume ratio of a blood flow pattern explained in the fifth embodiment may be used.

The respective processing functions included in the processing circuitry 250 have so far been explained. For example, the processing circuitry 250 is implemented by a processor. In this case, the respective processing functions described above are stored in the storage 120 in a form of computer-executable programs. The processing circuitry 250 implements functions corresponding to the respective programs by reading and executing the respective programs stored in the storage 120. In other words, the processing circuitry 250 is to have the respective functions illustrated in FIG. 9 in a state in which the processing circuitry 250 has read the respective programs.

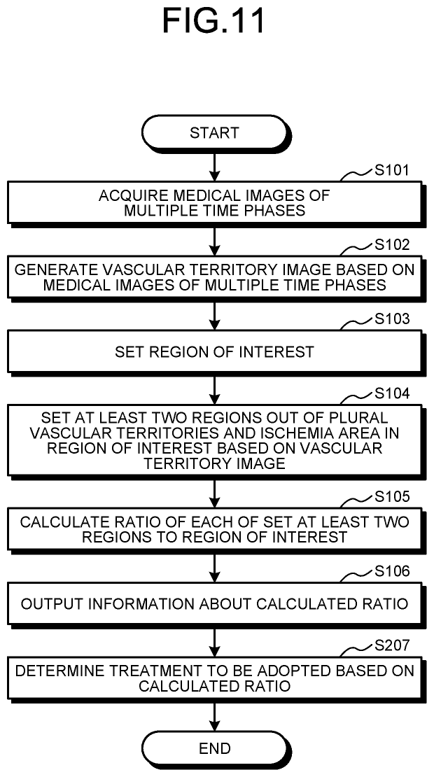

FIG. 11 is a flowchart illustrating a procedure of processing performed by the processing circuitry 250 according to the seventh embodiment.

For example, as illustrated in FIG. 11, the processing circuitry 250 performs processing similar to steps S101 to S106 in FIG. 3.

Subsequently, the processing circuitry 250 determines a treatment to be adopted to a subject tissue of a subject based on a calculated ratio between each of at least two regions and a region of interest (step S207). This step is a step corresponding to the determining function 257. For example, the processing circuitry 250 performs this step by reading and executing a program corresponding to the determining function 257 from the storage 120.

As described above, in the seventh embodiment, the determining function 257 determines a treatment to be adopted to a subject tissue of a subject based on a ratio between each of at least two regions to a region of interest. With such a configuration, a treatment method for a subject tissue of a subject can be determined more appropriately.

### OTHER EMBODIMENTS

In the embodiments described above, a case in which a subject tissue to be diagnosed is brain has been explained as an example, but embodiments are not limited thereto. For example, the medical image processing apparatus explained in the embodiments described above can be applied similarly to a case in which the subject tissue is other organs. Examples of organs herein include heart.

Moreover, in the embodiments described above, a case in which the first setting function 153 sets a region of interest by using the vascular territory image, the medical images of multiple time phases, the healthy vascular-territory image, or the perfusion image has been explained as an example, embodiments are not limited thereto. For example, the first setting function 153 may be configured to accept an operation to specify a range to be a region of interest on an image showing a subject tissue through the input interface 130, and to set the accepted range as the region of interest.

Furthermore, the configuration of the medical image processing apparatus explained in the above embodiments can be applied to a medical image diagnostic apparatus also. In this case, processing circuitry included in a console device or the like of the medical image diagnostic apparatus includes the acquiring function, the generating function, the first setting function, the second setting function, the calculating function, the output function, and the determining function described above.

Moreover, in the embodiments described above, a case in which the acquiring function, the generating function, the first setting function, the second setting function, the calculating function, the output function, and the determining function in the present specification are implemented by the acquiring function, the generating function, the first setting function, the second setting function, the calculating function, the output function, and the determining function of the processing circuitry, respectively has been explained as an example, but embodiments are not limited thereto. For example, the acquiring function, the generating function, the first setting function, the second setting function, the calculating function, the output function, and the determining function in the present specification may be implemented by hardware only, software only, or a combination of hardware and software implementing the same functions, in addition to by using the acquiring function, the generating function, the first setting function, the second setting function, the calculating function, the output function, and the determining function described in the embodiments.

Furthermore, in the embodiments described above, a case in which the processing circuitry is implemented by a single unit of processor has been explained as an example, but embodiments are not limited thereto. For example, the processing circuitry may be configured by combining multiple independent processors, to implement the respective processing functions by the respective processors executing programs. Moreover, the respective functions of the processing circuitry may be implemented by a single or multiple processing circuities in an appropriately distributed or integrated manner. Furthermore, the respective processing functions of the processing circuitry may be implemented by a combination of hardware, such as a circuit, and software. Moreover, a case in which the programs corresponding to the respective processing functions are stored in a single unit of the storage has been explained as an example, but embodiments are not limited thereto. For example, the respective programs corresponding to the respective processing functions may be stored in plural storages in a distributed manner, and the processing circuitry may be configured to read and execute the respective programs from the respective storages.

Moreover, a term “processor” used in the above explanation signifies a circuit, such as a central processing unit (CPU), a graphical processing unit (GPU), an application specific integrated circuit (ASIC), a programmable logic device (for example, simple programmable logic device (SPLD), complex programmable logic device (CPLD)), and a field programmable gate array (FPGA). Instead of storing a program in the storage, it may be configured to directly install a program in a circuit of the processor. In this case, the processor reads and executes the program installed in the circuit, to implement the function. Furthermore, the respective processors of the present embodiment are not limited to be configured such that each processor is implemented by one circuit, but may be configured to combine plural independent circuits to form one processor, to implement the function.

The program executed by the processor is installed in a read only memory (ROM), a storage, and the like to be provided. This program may be recorded in a computer-readable non-transient recording medium, such as a compact disk read-only memory (CD-ROM), a flexible disk (FD), a compact disk-recordable (CD-R), and a digital versatile disk (DVD), in a file in an installable format or an executable format by these apparatuses, to be provided. Moreover, this program may be stored in a computer that is connected to a network, such as the Internet, and may be provided or distributed by being downloaded through the network. For example, this program is configured with modules including the respective processing functions described above. As actual hardware, a CPU reads and executes the program from a storage medium, such as a ROM, and the respective modules are thereby loaded on a main storage device to be generated on the storage device.

Furthermore, in the embodiments and modifications described above, the respective components of the respective apparatuses illustrated are of functional concept, and it is not necessarily required to be configured physically as illustrated. That is, specific forms of distribution and integration of the respective apparatuses are not limited to the ones illustrated, and all or some thereof can be configured to be distributed or integrated functionally or physically in arbitrary units according to various kinds of loads, usage conditions, and the like. Furthermore, as for the respective processing functions performed by the respective apparatuses, all or an arbitrary part thereof can be implemented by a CPU and a computer program that is analyzed and executed by the CPU, or can be implemented as hardware by wired logic.

Moreover, out of the respective processing explained in the embodiments and modifications described above, all or some of processing explained to be performed automatically can also be performed manually, or all or some of processing explained to be performed manually can be performed automatically by a publicly-known method. In addition, the processing procedure, the control procedure, the specific names, information including various kinds of data and parameters can be changed arbitrarily unless otherwise specified.

All kinds of data handled in the present specification are digital data typically.

According to at least one embodiment explained above, a quantitative indicator of a collateral circulation that is directly connected to a treatment decision and a prognosis can be presented.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus, comprising:
processing circuitry configured to:
acquire medical images of a plurality of time phases of a subject tissue of a subject;
generate a vascular territory image showing a plurality of vascular territories included in the subject tissue based on the medical images of multiple time phases;
set a region of interest in the subject tissue;
set at least two regions out of the vascular territories and an ischemia area in the region of interest based on the vascular territory image;
calculate a ratio of each of the at least two regions to the region of interest; and
output information about the ratio.

2. The medical image processing apparatus according to claim 1, wherein
the processing circuitry is further configured to calculate a volume ratio of each of the at least two regions to the region of interest, as the ratio of each of the at least two regions to the region of interest.

3. The medical image processing apparatus according to claim 1, wherein
the subject tissue is brain, and
the processing circuitry is further configured to set the region of interest in a hemisphere including the ischemia area out of two hemispheres of the brain in the vascular territory image.

4. The medical image processing apparatus according to claim 3, wherein
the processing circuitry is further configured to identify an entire territory of a hemisphere on an opposite side to the hemisphere including the ischemia area by using at least one of the medical images of multiple time phases, and set an inverted territory of the identified territory relative to a median plane of the brain as the region of interest.

5. The medical image processing apparatus according to claim 3, wherein
the processing circuitry is further configured to detect a position of the ischemia area in the hemisphere including the ischemia area by using the vascular territory image, identify a vascular territory including a position corresponding to the position of the ischemia area in a hemisphere on an opposite side to the hemisphere including the ischemia area, and set an inverted territory of the identified vascular territory relative to a median plane of the brain as the region of interest.

6. The medical image processing apparatus according to claim 3, wherein
the processing circuitry is further configured to
acquire a healthy vascular-territory image showing a vascular territory in a healthy condition, and
detect a position of the ischemia area in the hemisphere including the ischemia area by using the vascular territory image, identify a vascular territory including a position of the ischemia area detected by the vascular territory image by using the healthy vascular-territory image, and set a region in which the identified vascular territory is mapped in the vascular territory image as the region of interest.

7. The medical image processing apparatus according to claim 3, wherein
the processing circuitry is further configured to
acquire a perfusion image of the subject, and
identify a low perfusion area in the hemisphere including the ischemia area by using the perfusion image, and set a region in which the identified low perfusion area is mapped in the vascular territory image as the region of interest.

8. The medical image processing apparatus according to claim 3, wherein
the processing circuitry is further configured to calculate a volume ratio of each of the at least two regions to the region of interest as a first volume ratio, calculate a volume ratio of each of the at least two regions included in the hemisphere on the opposite side to the hemisphere including the ischemia area to the entire territory of the hemisphere on the opposite side as a second volume ratio, and calculate a difference between the first volume ratio and the second volume ratio for each corresponding regions.

9. The medical image processing apparatus according to claim 3, wherein
the vascular territories are a middle cerebral artery territory, an anterior cerebral artery territory, and a posterior cerebral artery territory.

10. The medical image processing apparatus according to claim 1, wherein
the processing circuitry is further configured to classify the at least two regions into a plurality of blood flow patterns including a anterograde perfusion area, a retrograde perfusion area, and an ischemia area, and calculate the ratio for each of the blood flow patterns.

11. The medical image processing apparatus according to claim 10, wherein
the processing circuitry is further configured to output the vascular territory image showing a region of at least one blood flow pattern out of the blood flow patterns.

12. The medical image processing apparatus according to claim 1, wherein
the processing circuitry is further configured to determine a treatment to be adopted to the subject tissue based on the ratio of each of the at least two regions to the region of interest.

13. A medical image processing method, comprising:
acquiring medical images of a plurality of time phases of a subject tissue of a subject;
generating a vascular territory image showing a plurality of vascular territories included in the subject tissue based on the medical images of multiple time phases;
setting a region of interest in the subject tissue;
setting at least two regions out of a plurality of regions including the vascular territories and an ischemia area in the region of interest based on the vascular territory image;
calculating a ratio of each of the at least two regions to the region of interest; and
outputting information about the ratio.
